(19) United States
(12) Patent Application Publication
(10) Pub. No.: US 2015/0238194 A1
(43) Pub. Date: Aug. 27, 2015

(54) HEMOSTASIS DEVICES AND METHODS UTILIZING MECHANICAL METHODS

(71) Applicant: Boston Scientific Scimed, Inc., Maple Grove, MN (US)

(72) Inventors: John Allen HINGSTON, Framingham, MA (US); Rachael Ann RHEAUME, Framingham, MA (US); Kenny Joe KING, Somerville, MA (US); Daniel Robert QUINN, Littleton, MA (US); Kerry L. GRANT, Northbridge, MA (US); Gary S. KAPPEL, Acton, MA (US); John B. GOLDEN, Norton, MA (US); Andrew CALABRESE, Cambridge, MA (US); Amie PRESTON, Groton, MA (US); Tara Elizabeth DELAND, Marlborough, MA (US); Ananya TANDON, Worcester, MA (US)

(21) Appl. No.: 14/625,781

(22) Filed: Feb. 19, 2015

Related U.S. Application Data
(60) Provisional application No. 61/943,736, filed on Feb. 24, 2014.

(51) Int. Cl. A61B 17/122; A61B 17/128

(52) U.S. Cl. CPC A61B 17/122; A61B 17/1285

(57) ABSTRACT

A device for causing hemostasis includes an elongated body extending from a first end to a second end, a first clipping anchor coupled to the first end of the elongated body and being movable between a radially contracted insertion configuration and a radially expanded tissue engagement configuration and a second clipping anchor coupled to the second end of the elongated body and being movable between a radially contracted insertion configuration and a radially expanded tissue engagement configuration. The first clipping anchor is movable along a longitudinal axis of the elongated body one of toward and away from the second clipping anchor.

### PRIORITY CLAIM

The present invention claims priority to U.S. Provisional Patent Application Ser. No. 61/943,736 field Feb. 24, 2014; the disclosure of which is incorporated herewith by reference.

### BACKGROUND

Pathologies of the gastro-intestinal (“GI”) system, the biliary tree, the vascular system and other body lumens are commonly treated through endoscopic procedures, many of which require active and/or prophylactic hemostasis to control bleeding. Physicians have become increasingly willing to perform aggressive interventional and therapeutic endoscopic procedures which increase the risk of perforating the wall of the GI tract or require closure of openings in tissue of the GI tract as part of the procedure. Current devices for hemostasis can be difficult and time-consuming and in some case, are inefficient depending on the type of perforation, condition or anatomy being treated. There is a need for a hemostasis device that can be utilized in an array of procedures while also minimizing the time and effort required to perform a hemostasis procedure.

### SUMMARY OF THE INVENTION

The present invention relates to a device for causing hemostasis comprising an elongated body extending from a first end to a second end, a first clipping anchor coupled to the first end of the elongated body and being movable between a radially contracted insertion configuration and a radially expanded tissue engagement configuration and a second clipping anchor coupled to the second end of the elongated body and being movable between a radially contracted insertion configuration and a radially expanded tissue engagement configuration. The first clipping anchor is movable along a longitudinal axis of the elongated body one of toward and away from the second clipping anchor.

### BRIEF DESCRIPTION OF THE DRAWINGS

FIG. 1 shows a first side view of a hemostasis device according to a first exemplary embodiment of the present invention in a first configuration;

FIG. 2 shows a side view of the hemostasis device of FIG. 1 in a second configuration;

FIG. 3 shows a cross-sectional view of the hemostasis device of FIG. 1 in a first operative configuration;

FIG. 4 shows a cross-sectional view of the hemostasis device of FIG. 1 in a second operative configuration;

FIG. 5 shows a cross-sectional view of the hemostasis device of FIG. 1 in a third operative configuration;

FIG. 6 shows a cross-sectional view of a hemostasis device according to a first alternate embodiment of the invention;

FIG. 7 shows a cross-sectional view of a hemostasis device according to a second alternate embodiment in an insertion configuration;

FIG. 8 shows a cross-sectional view of the hemostasis device of FIG. 7 in a tissue-engaging configuration;

FIG. 9 shows a perspective view of the hemostasis device of FIG. 7 in the tissue-engaging configuration;

FIG. 10 shows a side view of a hemostasis device according to a third alternate embodiment of the invention;

FIG. 11 shows a perspective view of the hemostasis device of FIG. 10 in a first operative configuration;

FIG. 12 shows a perspective view of the hemostasis device of FIG. 10 in a second operative configuration;

FIG. 13 shows a perspective view of the hemostasis device of FIG. 10 in a third operative configuration;

FIG. 14 shows a perspective view of the hemostasis device of FIG. 10 in a fourth operative configuration;

FIG. 15 shows a partial cross-sectional view of the of the hemostasis device of FIG. 10 in the first operative configuration;

FIG. 16 shows a partial cross-sectional view of the of the hemostasis device of FIG. 10 in the second operative configuration;

FIG. 17 shows a first partial cross-sectional view of the of the hemostasis device of FIG. 10 in the third operative configuration;

FIG. 18 shows a second partial cross-sectional view of the of the hemostasis device of FIG. 10 in the third operative configuration; and

FIG. 19 shows a side view of a hemostasis device according to a fourth alternate embodiment of the invention.

### DETAILED DESCRIPTION

The present invention may be further understood with reference to the following description and the appended drawings, wherein like elements are referred to with the same reference numerals. Embodiments of the invention are directed to clipping devices having first and second anchors configured to capture tissue therebetween, the clipping devices being movable from a reduced profile insertion configuration to a radially expanded tissue engagement configuration in which the first and second anchors expand radially outward to engage target tissue to hold the clipping device in a desired location relative to target tissue. In a first exemplary embodiment, the clipping device includes an elongated body having a first anchor on a first end and a second anchor on a second end thereof. The first and second anchors in this embodiment are umbrella-like structures, each including a plurality of ribs extending from the respective end with a mesh or fabric material extending over the ribs. In an operative configuration, the first end including the first anchor member is inserted through a perforation in target tissue from a first side of the perforation and deployed to an expanded configuration on a second side of the perforation opposite the first side. The second anchor member is then deployed to the expanded configuration on the first side of the perforation to lock the clipping device in place. The body is structured to be extendable from a first shorter length to a second longer length and may be spring loaded and biased toward the first length. Thus, upon deployment of the first and second anchors from an endoscope or other insertion instrument, the first and second anchors are compressed by the spring bias against proximal and distal walls of the tissue adjacent the perforation, effectively sealing the perforation. In accordance with another embodiment, the first and second anchors are formed with corresponding threaded portions to threadedly engage one another across the perforation, as will be described in greater detail later on. In yet another embodiment, the first anchor is positioned on a distal end of an elongated rod. A second anchor is constrained to slide along the elongated rod in only one direction by a ratchet mechanism, as will also be described in greater detail later on. It should be noted that the terms “proximal” and “distal,” as used herein, are intended to refer to a direction toward (proximal) and away from (distal) a user of the device.

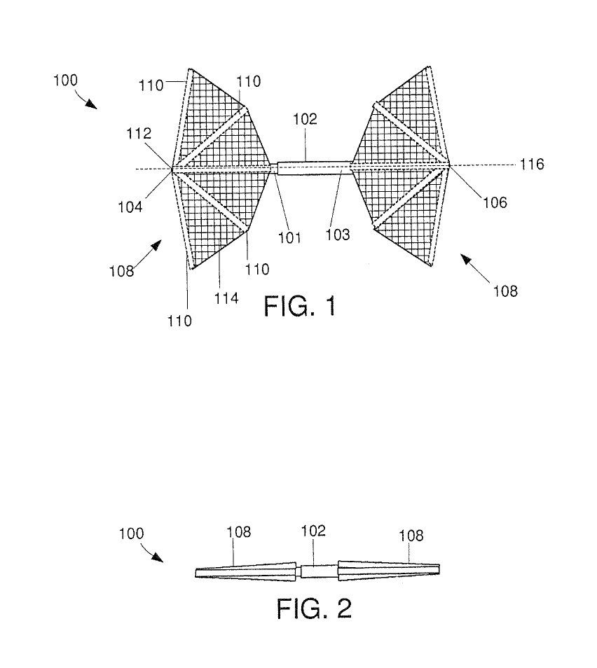

As shown in FIGS. 1-2, a hemostasis device 100 according to an exemplary embodiment of the invention includes an elongated body 102 extending from a first end 104 to a second end 106. An umbrella-type collapsible tissue engagement mechanism 108 is provided at each of the first and second ends 104, 106. The tissue engagement mechanism 108 is movable from a collapsed configuration shown in FIG. 2 to an expanded configuration, shown in FIG. 1. The tissue engagement mechanism 108 includes a plurality of ribs 110 extending away from a connection 112 at the first and second ends 106. It is noted that although only three ribs 110 are depicted in FIGS. 1-2, any number of ribs 110 may be employed without deviating from the scope of the disclosure. For example, a greater number of ribs 110 may be used to provide increased strength resistance to the tissue engagement mechanism. In one embodiment, each of the tissue engagement mechanisms 108 includes 2-5 ribs. The length of the ribs 110 may, for example, be selected to be at least as great as or greater than a radius of a circle enclosing a perforation in the wall of the GI tract, thereby ensuring that the entirety of the perforation is enclosed by the tissue engagement mechanism. The tissue engagement mechanism 108 is biased to the expanded configuration to permit automatic deployment thereof as the mechanism 108 is extended out of an insertion instrument, as will be described in greater detail with respect to the method below. In one embodiment, the ribs 110 extend perpendicular to the axis 116 in the biased expanded configuration. It is noted, however, that the ribs 110 may extend at any angle relative to the axis 116 without deviating from the scope of the invention. In another embodiment, the ribs 110 may expand to an angle greater than 90 degrees relative to the axis 116. The ribs 110 may be formed with a heat set at the connection 112, the heat set preventing the ribs 110 from extending more than a predetermined angle away from the closed configuration. A mesh 114 is provided over the ribs 110 to provide additional holding strength to the tissue engagement mechanism 108. In an alternate embodiment, the mesh 114 may be replaced with an adhesive material, tissue, bio-genetically created tissue, semi-solid glue, gauze, nylon string or other material known in the art. The mesh 114 is preferably a dense mesh of biocompatible material (e.g., a fabric) secured to the ribs 110 using an attachment method known in the art. For example, the mesh 114 may be secured to each of the ribs by sewing a thread through the mesh and around each of the ribs 110. Alternately, an adhesive or other known attachment means known in the art may be used. The material of the mesh 114 is sufficiently rigid to apply a compressive force to adjacent portions of tissue while also being sufficiently flexible to fold into itself when the hemostasis device 100 is moved to the collapsed configuration of FIG. 2.

In one embodiment, a plurality of hemostasis devices 100 may be used in conjunction with one another to close a larger opening by passing the device through a first portion of tissue adjacent to one side of the opening and then through a second portion of tissue adjacent the other side of the opening. The first tissue engagement mech 108 may then be opened on the far side of the second portion of tissue and the second tissue engagement mechanism would be opened on the proximal side of the first portion of tissue. Additional devices 100 may then be deployed similarly along the length of the opening to draw the first and second sides thereof closed.

The body 102 is extendible and retractable along an axis 116 extending through the first and second ends 104, 106. The body 102 is formed as a spring-loaded body which is biased to a first shorter length and may be expanded to a longer length in an operative configuration—e.g., due to an expanding pressure applied by walls of the target tissue. The body 102 comprises first and second elongated tubes 101, 103 axially slidable relative to one another. Specifically, the first tube 101 is slidably received within the second tube 103. The body 102 is movable from a shorter length wherein the first tube 101 is seated partially or fully within the second tube 103 to a longer length wherein the first tube 101 moves partially out of the second tube 103.

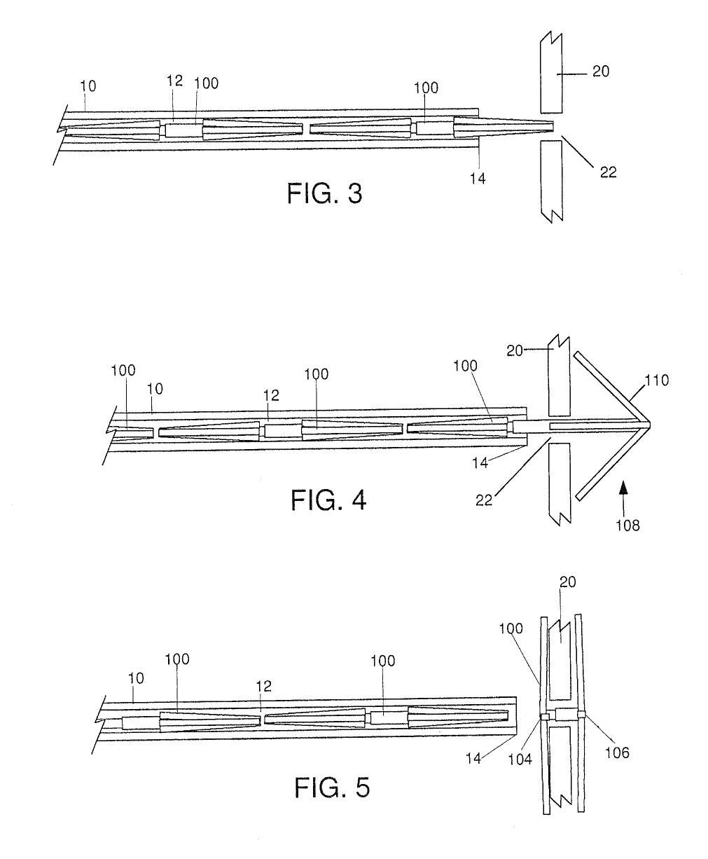

In accordance with an exemplary method according to the invention, the hemostasis device 100 is loaded into a working channel 12 of an endoscope or other insertion apparatus 10 in the collapsed configuration. In one embodiment, a plurality of devices 100 are sequentially loaded in the working channel 12 to permit the deployment of multiple devices 100 without having to remove the endoscope 10 from the body or reload the endoscope 10 during use, as those skilled in the art will understand. Walls of the working channel apply a radially constrictive pressure to the hemostasis device 100 to prevent the ribs 110 from opening to their expanded configuration. Furthermore, the spring-loaded bias of the ribs 110 engages the walls of the working channel 12 with sufficient force to prevent inadvertent advancement thereof out of the working channel 12. Rather, only a distally directed pushing force, as described in greater detail hereinafter, is sufficient to counter the radially expansive force applied between the ribs 110 and the working channel 12 to cause a distal advancement of the hemostasis device 100 therein. In an exemplary embodiment, adjacent ones of the devices 100 directly abut one another and are advanced distally through the working channel 12 in unison by, for example, a pusher member (not shown) located proximally of a proximal most one of the devices 100. The pusher member (not shown) extends to a proximal end including an actuator located outside of the body and accessible to a physician or other user in an operative configuration. In the next step, the endoscope is advanced to a target position so that a distal end 14 of the endoscope 10 is positioned adjacent a target tissue site. Specifically, the distal end 14 is positioned through a perforation 22 in the tissue 20 (e.g., through a wall of the GI tract, etc.) so that, as the hemostasis device 100 extends out of the endoscope 10, the hemostasis device 100 moves through the perforation 22, as will be described in greater detail hereinafter. In another embodiment, the distal end 14 may be positioned over the target tissue, the device 100 piercing the tissue before deploying on an opposite side thereof. As shown in FIGS. 3-4, the actuator is then moved to advance the hemostasis device 100 out of the working channel 12 so that a first one of the tissue engagement mechanisms 108 moves through the perforation 22, as shown in FIGS. 3-4. When the first one of the tissue engagement mechanisms 108 has been advanced out of the working channel 12, the ribs 110 are freed to return under their bias to the expanded configuration, as shown in FIG. 4. The endoscope 10 may then be retracted a short distance so that the expanded tissue engagement mechanism 108 abuts against an opposing wall of the perforation, as shown in FIG. 4. That is, the device 100 is coupled to a delivery system (not shown) including the pusher member (not shown) in the endoscope 10. As the endoscope 10 is advanced proximally and distally, the device 100 also moves proximal and distally. After the endoscope 10 has been withdrawn proximally to a proximal side of the perforation, the hemostasis device 100 is advanced further distally out of the working channel 12 until the second tissue engagement mechanism 108 exits the working channel 12 and springs to the expanded configuration. Furthermore, the spring-loaded construction of the body 102 retracts the body 102 so that the first and second the tissue engagement mechanisms 108 are drawn toward one another into contact with opposing walls of the perforation, as shown in FIG. 5. The endoscope 10 may then be repositioned as necessary and the above method repeated as many times as necessary to perform a target procedure.

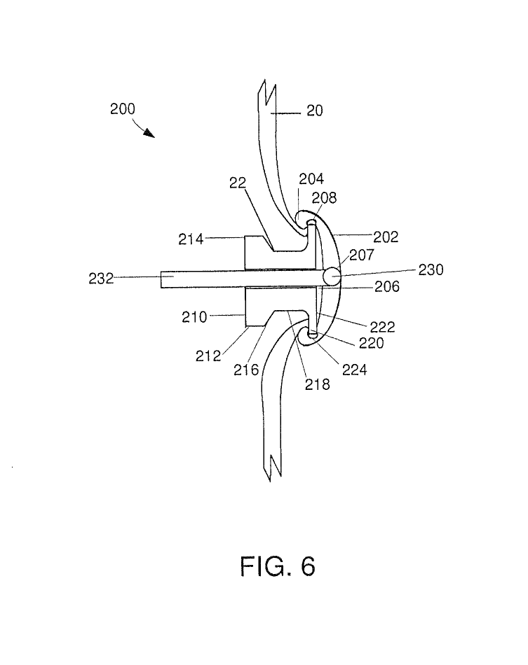

FIG. 6 depicts a hemostasis device 200 according to another embodiment of the invention. The device 200 includes a first collapsible portion 202 insertable through the perforation in the tissue 20 in a collapsed configuration and expandable to the configuration depicted in FIG. 6. As will be described in greater detail with respect to the exemplary method below, a distal opening of an endoscope or other insertion device is positioned over the perforation 22 in the tissue 20 so that, as the hemostasis device 200 is advanced out of the working channel 12 of the endoscope 10, the collapsible portion 202 extends through the perforation and moves to the expanded configuration on a distal side of the perforation 22. The collapsible portion 202 is formed with a substantially concave dome having a rim 204 and defining a cavity 206 therein, walls of the cavity 206 including threading 208 formed to engage corresponding threading 224 on a locking portion 210, as will be described in greater detail below. The rim 204 is formed by folding in a periphery of the collapsible portion 202 so that the portion of the collapsible portion 202 contacting the tissue 20 is rounded and includes no sharp edges. The collapsible portion 202 in an embodiment of the invention is biased to assume the shape depicted in FIG. 6 and may be folded during insertion thereof through the endoscope (not shown). The collapsible portion 202 includes an opening 207 formed therein to removably receive an enlargement 230 formed at a distal end of a control wire 232. As will be described in greater detail with respect to the exemplary method below, the control wire 232 guides placement of the collapsible portion 202 over a target portion of tissue and may be separated from the collapsible portion 202 after a target procedure has been completed.

The hemostasis device 200 further comprises the locking portion 210 which lockingly engages the collapsible portion 202 in an operative configuration. The locking portion 210 is formed with an increased diameter head 212 at a first end 214, the head 212 having a diameter at least greater than a diameter of a circle enclosing the perforation 22. A bottleneck portion 216 extends from the head 212 to an elongated body 218 which is situated through the perforation 22 in an operative configuration. The bottleneck portion 216 gradually tapers down in diameter from the head 212 to the reduced diameter of the body 218. An increased diameter lip 220 is formed at a second end 222 of the locking portion 210, the lip 220 having threading 224 formed to threadedly engage threading 208 of the collapsible portion 202. The locking portion 210 includes a channel 217 extending therethrough to receive the control wire 232, a diameter of the channel 207 being greater than a diameter of the enlargement 230. The collapsible portion 202 and locking portion 210 according to the embodiment may be formed of a semi-rigid or rigid biocompatible material including, but not limited to, metals and polymers. The collapsible portion 202 may be formed of flexible or elastic biocompatible materials such as NiTi or polymers such as nylon, pebax or a biodegradable polymer.

In accordance with an exemplary method according to the present embodiment, an endoscope or other insertion device (not shown) is fitted with one or more hemostasis devices 200, wherein the collapsible portion 202 is housed within the working channel of the endoscope (not shown) in a collapsed, reduced profile configuration. In another embodiment, the device 200 is made small enough to fit through the endoscope without the need for folding, etc. In another embodiment, the device 200 may be an over-the-scope device, as those skilled in the art will understand. The device 200 is loaded into the endoscope such that the collapsible portion 202 is located distally of the locking portion 210, both of which are coupled to the control wire 232. A distal end of the endoscope is positioned over the perforation 22 and an actuator (not shown) coupled to a proximal end of the control wire 232 is actuated to advance the collapsible portion 202 out of the working channel. As the collapsible portion 202 moves out of the working channel 202 and through the perforation 22, the collapsible portion 202 expands to the configuration depicted in FIG. 6. Specifically, the collapsible portion 202 is situated within the working channel in a configuration selected so that, when moved distally thereoutof and expanded, the rim 204 is oriented toward the tissue bordering the perforation 22. This orientation may be maintained by the control wire 232 coupled to the collapsible portion 202. Once deployed out of the endoscope, the control wire 232 or other retaining means coupled to the collapsible portion 202 is retracted proximally to ensure that the rim 204 is fully seated over the tissue 20 bordering the perforation 22. A distally directed force is then applied to the locking portion 204 to force the distal portion out of the distal end of the working channel and adjacent to the collapsible portion 202. The lip 220 and body 218 are advanced through the perforation 22 while the increased diameter of the head 212 and bottleneck 216 prevent insertion thereof through the perforation 22. Once the lip 220 is seated within the cavity 206, the locking portion 210 is rotated to cause threads 224 of the lip 220 to threadedly engage threads 208 of the cavity 206, capturing tissue therebetween. The rotation may be achieved via a pull-string wound about the locking portion 210, wherein pulling the pull-string (not shown) proximally rotates the locking portion 210 as the pull-string unravels. In another embodiment, the rotation may be achieved via a gear mechanism coupled to the locking portion, as those skilled in the art will understand. In yet another embodiment, a separate instrument may be inserted through the endoscope and manipulated to cause a rotation of the locking portion. Once a predetermined number of turns has been applied to the locking portion 210, the retaining mechanism (not shown) coupled to the hemostasis device 200 may be disengaged therefrom. Specifically, once the device 200 has been locked to the tissue, a proximally directed force is applied to the control wire 232. Once the force exceeds a predetermined threshold, the enlargement dislodges from the recess 207 to permit the control wire 232 and enlargement 230 to be withdrawn from the body. In another embodiment, the proximally directed force causes the control wire 232 to break off from the enlargement 232, so that the enlargement remains within the recess 207 and the control wire 232 is removed from the body. This process may be repeated as necessary to conform to the requirements of a particular procedure.

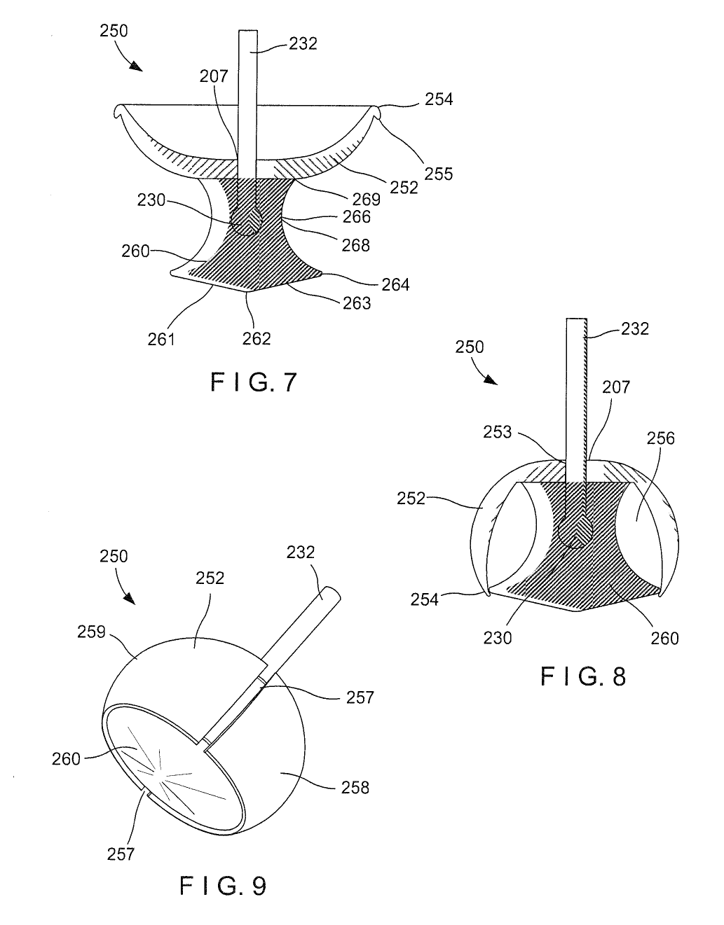

FIGS. 7-9 depict a hemostasis device 250 according to another embodiment, the device 250 being formed substantially similar to the device 200 except as noted below. Whereas the collapsible portion 202 and locking portion 210 of the device 200 are adapted to threadedly engage one another, a collapsible portion 252 and locking portion 260 are adapted to engage each other with a pressure fit. Specifically, the collapsible portion 202 is formed of a flexible material similar to the collapsible portion 202 and is biased toward a concave dome shape defining a cavity 256 therein, as depicted in FIGS. 8 and 9. An outer profile of the collapsible portion 252 is substantially semi-spherical and extends from an apex 253 to a rim 254 having a radially protruding lip 255 extending therefrom. In an operative configuration, the lip 255 lockingly engages a first end 264 of the locking member 260. During insertion, the collapsible portion is moved to a outwardly folded configuration in which the rim 254 is moved proximally so that the rim 254 is located proximally of the apex 253, as shown in FIG. 7. The collapsible portion 252 may include one or more slots 257 extending from the rim 254 toward the apex 253 but terminating short of the apex 253, the slots 257 defining first and second deflectable portions 258, 259. As those skilled in the art will understand, the slots 257 aid in radial expansion of the collapsible portion 252 to permit movement thereof between the insertion configuration and a tissue engagement configuration. The collapsible portion 202 also includes an opening 207 to removably receive the enlargement 230 formed at a distal end of the control wire 232. As will be described in greater detail with respect to the exemplary method below, the control wire 232 guides placement of the collapsible portion 252 over a target portion of tissue and may be separated from the collapsible portion 252 after a target procedure has been completed.

The locking portion 260 is formed with an increased diameter head 262 at a first end 264. Distally facing walls 261, 263 are angled to converge at the first end 264 to guide insertion of the locking portion 260 through target tissue. Similar to the locking portion 210, a bottleneck portion 266 extends from the head 262 and defines a reduced diameter body 268 of the locking portion 260, the diameter increasing back from the body 268 to a second end 269. An opening 270 is formed in the locking portion 260 to receive the control wire 232 therethrough, the opening 270 including an enlarged end 272 sized to receive the enlargement 230 of the control wire 232 therein.

In accordance with an exemplary method according to the present embodiment, an endoscope or other insertion device (not shown) is fitted with one or more hemostasis devices 250, with the collapsible portion 252 housed within the working channel of the endoscope (not shown) in a proximally retracted configuration as shown in FIG. 7. In the insertion configuration, the locking portion 260 is positioned distally of the collapsible portion 252 so that, as the device 250 is advanced distally out of the endoscope, the locking portion 260 extends out of the working channel first. The control wire 232 is then actuated to advance the locking portion 260 and collapsible portion 252 out of the working channel. A distally directed force is then applied to the collapsible portion 252 to force the rim 254 in a distal direction toward the locking portion 260 to assume a tissue engaging configuration. As the first and second deflectable portions 258, 259 are moved distally, tissue located between the collapsible portion 262 and the locking portion 260 is lockingly captured between the inner wall of the collapsible portion 252 and the outer wall of the locking portion 260. In this configuration, the lip 255 lockingly extends around and lockingly engages the first end 264 of the locking portion 260. The bottleneck 266 is sized to permit the tissue to be housed therein in the tissue engaging configuration. Once the device 250 has been locked to the tissue, a proximally directed force is applied to the control wire 232. Once the force exceeds a predetermined threshold, the enlargement dislodges from the opening 270 to permit the control wire 232 and enlargement 230 to be withdrawn from the body. In another embodiment, the proximally directed force may cause the control wire 232 to break off from the enlargement 232, so that the enlargement remains within the opening 270 and the control wire 232 is removed from the body.

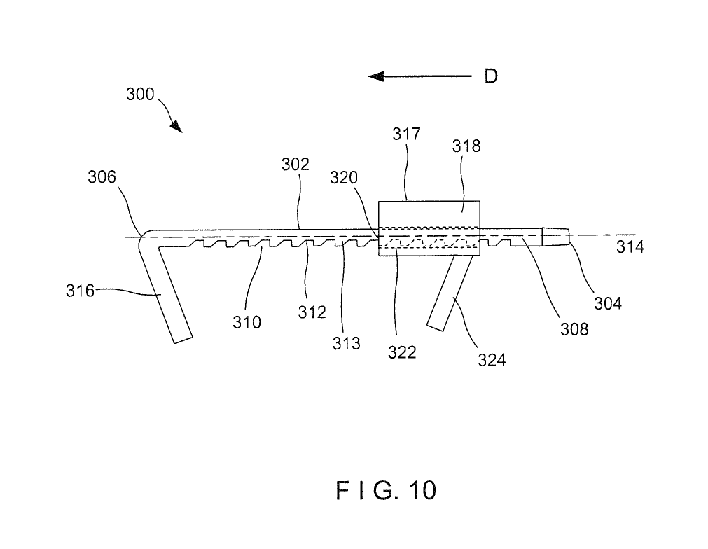

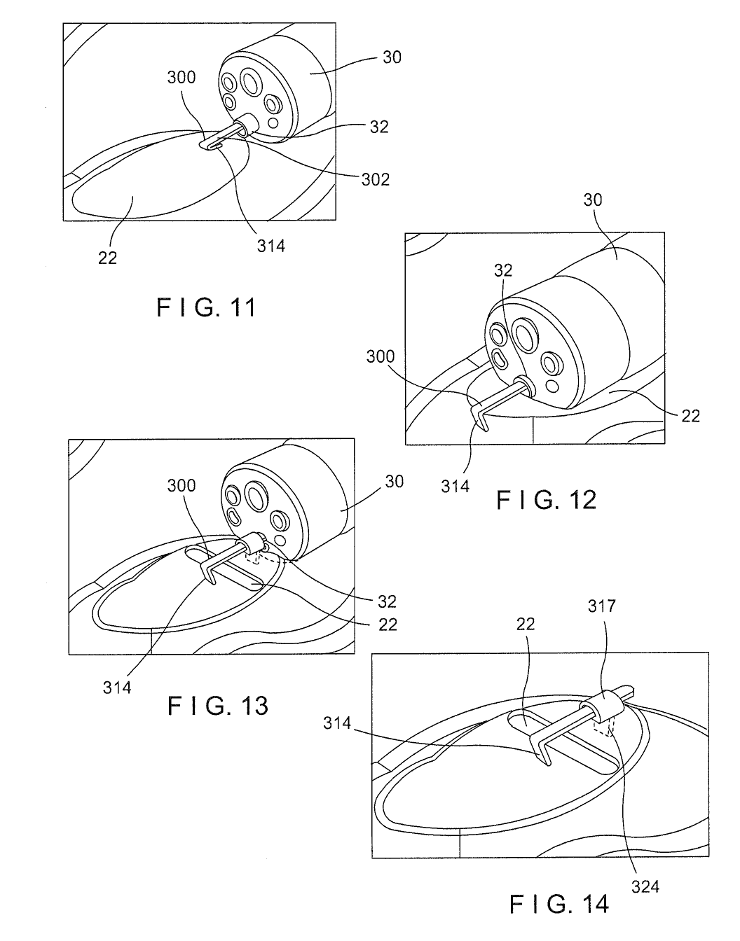

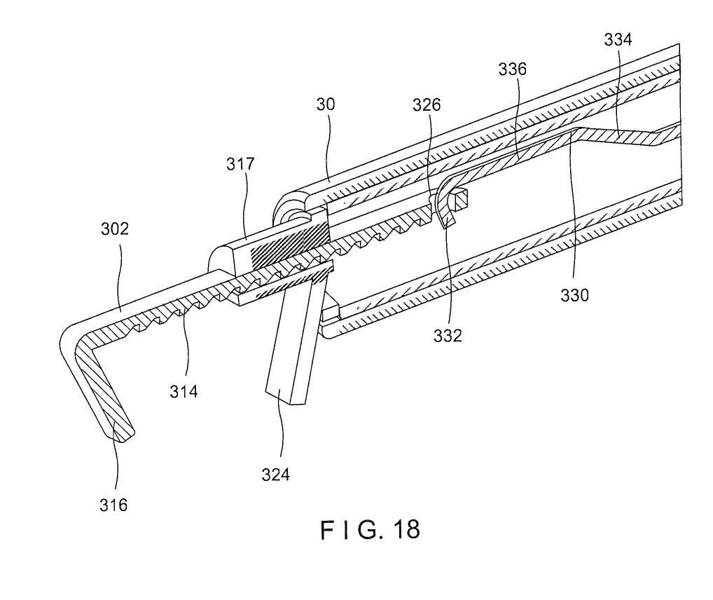

FIGS. 10-18 depict a hemostasis device 300 according to yet another embodiment. The hemostasis device 300 includes a straight, elongated body 302 extending from a proximal end 304 to a distal end 306. Components of the hemostasis device 300 may be formed of one or more of a metal, plastic, ceramic or a bioabsorbable material. A predetermined length of a first side wall 308 of the body 302 includes a plurality of ramped recesses 310. The recesses 310 include a first wall 312 extending substantially orthogonal to a longitudinal axis 314 of the body 302 and a second wall 313 enclosing an angle of between 0 and 90 degrees with the first wall 312. As will be described in greater detail later on, the ramped recesses 310 permit a ratcheting engagement of the body with a second anchor 324. In an exemplary embodiment, the body 302 has a rectangular cross-section, as seen more clearly in FIGS. 11-14. It is noted, however, that any other cross-sectional shape (e.g., circular, elliptical, square, triangular, etc.) may be used without deviating from the scope of the invention. The distal end of the body 302 further comprises a first anchor 316 extending out of the first wall 312 in a proximal direction and enclosing an acute angle therewith. The angle enclosed between the first wall 312 and the first anchor 316 may, for example, be 10-15 degrees and may be as high as 90 degrees depending on the design and functional requirements for a target procedure. The functional requirements may depend on, for example, tissue type, area, density and thickness. The design may depend on a shape, thickness, material and spring properties of the body 302 and first anchor 316. The first anchor 316 may optionally include any number of serrations, barbs or other tissue engaging features to enhance an engagement thereof with the tissue. In a preferred embodiment, a width of the first anchor 316 is the same as a width of the body 302. In another embodiment, the first anchor 316 may be formed with a width enlarged or reduced with respect to the width of the body 302. A width of the body 302 and first anchor 316 is greater than a thickness thereof, as shown in FIGS. 11-14 so that a profile of the device 300 may be reduced while still grasping a sufficient portion of tissue to ensure effective hemostasis closure, as will be described in greater detail with respect to the exemplary method below. The first anchor 316 is movable relative to the body 302 between a first configuration enclosing a first angle with the body 302, as shown in FIG. 12, and a second configuration enclosing a second angle with the body 302, the second angle being smaller than the first angle, as shown in FIG. 11. The reduced profile of the second configuration permits the first anchor 316 to be folded against the body to permit insertion thereof through a working channel 32 of an endoscope 30. The first anchor 316 is biased to the first configuration depicted in FIG. 10 so that, as the first anchor 316 moves out of the working channel 32 of the endoscope, the first anchor 316 automatically moves to the first configuration.

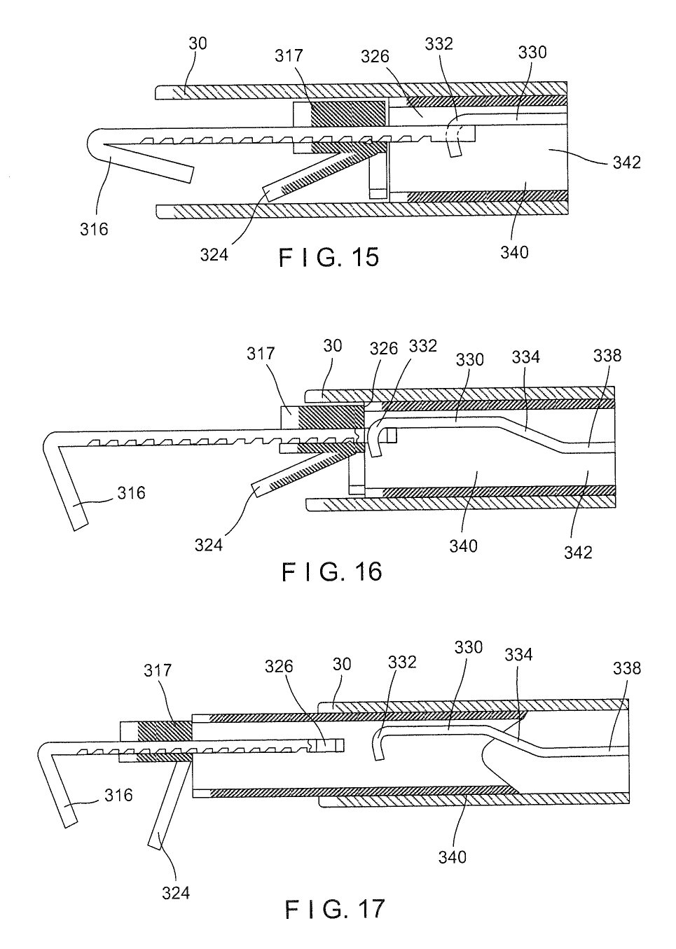

As shown in FIGS. 15-18, an opening 326 extends through the body 302 adjacent to the distal end 304. The opening 326 may be circular, oblong, square, or any other shape and is enclosed on all sides by the body 302. The opening 326 is sized to removably engage a control wire 330 extending through the endoscope 30 so that a proximal end of the control wire 330 remains accessible to and actuatable by a surgeon or other user to control axial movement of the control wire 330 along a longitudinal axis of the endoscope 30. The control wire 330 is formed as an elongated wire having a curved hook 332 at a distal end thereof, the hook 332 being received through the opening 326 to lockingly engage the hemostasis device 300. A distal portion of the control wire 330 includes an angled wall 334 formed such that a distal length 336 of the control wire 330 is laterally offset from a proximal portion 338 thereof. This configuration permits the hook 332 to extend along a side of the hemostasis device 300 and to be received through the opening 326. As will be described in greater detail later on, once the device 300 has been placed in a desired orientation in the body, the hook 332 is separated from the opening 326 to deploy the device 300 in situ. It is noted that although the embodiment is depicted with an opening 326 removably engaging the control wire 330, any other engagement mechanism may be used without deviating from the scope of the invention. For example, the body 302 may include a notch that only extends partially therethrough to removably engage the control wire 330. In an operative configuration, the control wire 330 engages the body 302 so that rotation of the control wire 330 rotated the hemostasis device 300 to control an orientation thereof relative to the target tissue.

The hemostasis device 300 further comprises a ratchet member 317 slidably received over the body 302. The ratchet member 317 includes a cylindrical body 318 having a channel 320 extending therethrough. A cross-sectional shape of the channel 320 is substantially rectangular to match a cross-sectional shape of the body 302. It is noted, however, that any shape of the channel 320 may be employed to conform to the shape of the body 302. A side wall 322 of the channel 320 positioned against the ramped recesses 310 in an operative configuration includes one or more protrusions (not shown) configured to ratchetly engage the ramped recesses 310. As those skilled in the art will understand, this ratcheted engagement permits movement of the ratchet member 317 only in a direction D. It is noted that although the present embodiment is described with respect to a ratchet mechanism, any other mechanism that permits one-way movement may be employed without deviating from the scope of the invention. In one embodiment, the ratchet mechanism 316 may be replaced with a rack and pinion mechanism, as those skilled in the art will understand. The ratchet member 317 further comprises a second anchor 324 angled toward the distal end 306 and enclosing an acute angle with the body 302. Similar to the first anchor 316, the second anchor is also movable from a first configuration enclosing a first angle with the body 302, as shown in FIG. 10, and a second configuration (not shown) enclosing a second angle with the body 302, the second angle being smaller than the first angle. Free ends of the first and second anchors 316, 324 may be sharpened to aid in penetration of tissue.

The endoscope 30 is also fitted with a pusher member 340 having an outer diameter equivalent to or marginally smaller than a diameter of the working channel 32. The pusher member 340 is formed as an elongated cylindrical element having a channel 342 extending therethrough to received the body 302 therethrough. The diameter of the channel 342 being smaller than a diameter of the ratchet member 317 such that the ratchet member 317 cannot be drawn therein. In an operative configuration, a proximal portion of the body 302 is received within the channel 342 while the ratchet member 317 is positioned distally thereof, as shown in FIGS. 15-18. The pusher member 340 extends to a proximal end accessible to a surgeon or other user to permit controlled advancement of the ratchet member 317 out of the endoscope 30. The pusher member 340 extend to a proximal end of the endoscope 30 accessible to a surgeon or other user. In an operative configuration, a proximal end (not shown) of the pusher member 340 is actuated to cause a corresponding movement of the pusher member 340 and control deployment of the ratchet member 317 into the tissue along the body 302.

In accordance with an exemplary method according to the invention, the hemostasis device 300 is loaded into the working channel 32 of the endoscope 30 in a collapsed configuration with each of the first and second anchor members 316, 324 folded toward the body 302 and the hook 332 of the control wire 330 received within the opening 326 in the hemostasis device 300. An actuation mechanism (not shown) is then manipulated to advance the pusher member 340 and, consequently, the control wire 330 and hemostasis device 300 distally. As the first anchor 316 is advanced out of the working channel 32, the first anchor 316 moves to the first configuration, as shown in FIGS. 12-13 and 16-17. The pusher member 340 is advanced distally until the first anchor 316 extends beyond a distal end of a perforation 22 or other target tissue region, as shown in FIG. 13. As those skilled in the art will understand, the endoscope 30 and hemostasis device 300 may then be withdrawn proximally a predetermined short distance to force the first anchor 316 to lockingly engage the target tissue. The pusher member 340 is then advanced further distally until engagement of the first anchor 316 with the tissue prevents the hemostasis device 300 from moving further distally into the tissue. Rather, the distal advancement of the pusher member 340 causes the ratchet member 317 to slide distally over the body 302. As the ratchet member 317 is advanced out of the working channel 32, the second anchor 324 moves to the first configuration and extends into a portion of tissue 20 located proximally of the perforation 22, as shown in FIGS. 14 and 17. The ratchet mechanism 317 is advanced distally along the body 302, causing the second anchor 324 to move toward the first anchor 316 and causing a corresponding movement of the proximal and distal walls adjacent the perforation or other target tissue. This movement of the tissue substantially closes the perforation 22. Once the physician or other user has confirmed, through endoscopic or other visualization, that the perforation 22 is substantially closed, the control wire 330 is manipulated (e.g., rotated, moved laterally away from the body 302, etc.) so that the curved hook 332 disengages the opening 326. The procedure may be repeated as necessary to conform to the requirements of a particular procedure.

It is noted that although the embodiment of FIGS. 10-18 depicts one hemostasis device 300 extending through the endoscope 30, any number of devices 300 may extend through the endoscope 30 without deviating from the scope of the invention. Specifically, once a single hemostasis device 300 has been deployed in the tissue, the control wire 330 and pusher member 340 may be withdrawn from a proximal opening (not shown) of the endoscope 30 located externally of the body in the operative configuration. An additional hemostasis device 300 may then be coupled to the control wire 330 and inserted into the proximal opening (not shown) of the endoscope, followed by the pusher member 340. The above method may be repeated any number of times to perform any number of tissue clipping procedures. In another embodiment, the endoscope 30 may be withdrawn from the body after performing a target procedure. An additional hemostasis device 300 may then be loaded into a distal opening of the endoscope 30. The endoscope may then be reinserted into the body to perform a target tissue clipping procedure. This method may also be repeated as often as required to conform to the requirements of a particular procedure.

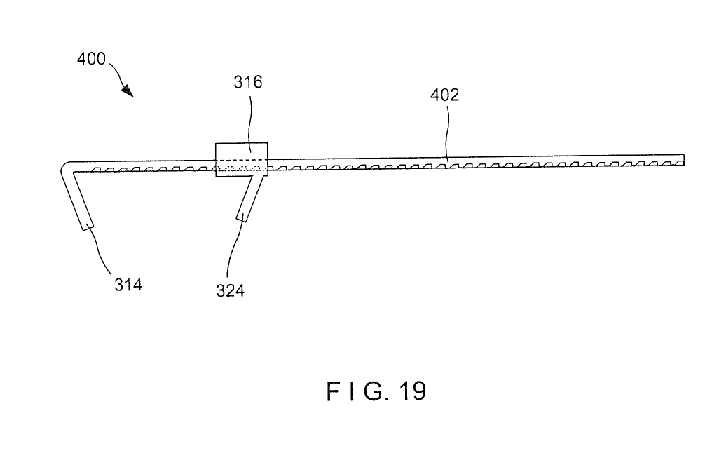

FIG. 19 depicts a device 400 according to another embodiment of the invention. The device 400 is formed substantially similar to the device 300, wherein like elements have been assigned like reference number. The hemostasis device 400 differs from the hemostasis device 300 in that the body 402 has a length enlarged relative to the body 302. As those skilled in the art will understand, the enlarged body 402 permits use of the hemostasis device 400 in closing larger wounds or perforations. It is noted that any length of the body 302, 402, first anchor 316 and second anchor 324 is envisioned within the scope of the invention and to conform to the requirements of a particular procedure.

It will be understood by those of skill in the art that individual features of the embodiments described above may be omitted and or combined to form alternate embodiments. Furthermore, it will be understood by those skilled in the art that various modifications can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A device for causing hemostasis, comprising;
an elongated body extending from a first end to a second end;
a first clipping anchor coupled to the first end of the elongated body and being movable between a radially contracted insertion configuration and a radially expanded tissue engagement configuration; and
a second clipping anchor coupled to the second end of the elongated body and being movable between a radially contracted insertion configuration and a radially expanded tissue engagement configuration;
wherein the first clipping anchor is movable along a longitudinal axis of the elongated body one of toward and away from the second clipping anchor.

2. The device of claim 1, wherein the first and second clipping anchors are formed as umbrella mechanisms.

3. The device of claim 2, wherein each umbrella mechanism comprises a plurality of elongated ribs connected to a common connection point and being pivotable relative thereto to move between the radially contracted and radially expanded configurations.

4. The device of claim 3, further comprises a covering provided over the plurality of ribs, the covering being one of a fabric and a mesh.

5. The device of claim 1, wherein the first and second clipping anchors are permanently attached to the first and second ends, respectively, of the body.

6. The device of claim 1, wherein the body is expandable from a first predetermined length to a second predetermined length.

7. The device of claim 6, wherein the body includes a first member slidably received within a channel of a second member.

8. The device of claim 1, wherein the second clipping anchor is slidable along the elongated body.

9. The device of claim 8, wherein the second clipping anchor is coupled to a ring, the ring being movable in only one direction along the elongated body in a direction extending toward the first clipping anchor.

10. The device of claim 9, wherein the ring is ratchetly coupled to the elongated body.

11. The device of claim 8, wherein the elongated body includes an opening formed to removably engage a control wire therein.

12. The device of claim 8, further comprising a pusher member moving the second clipping anchor distally relative to the first clipping anchor.

13. A device for causing hemostasis, comprising;
a first clipping member removably coupleable to a free end of a control wire and having a concave dome-shaped body defining a cavity therein, the first clipping member extending from a tip to a rim; and
a second clipping member coupled having an opening formed to slidably receive the control wire therethrough and being movable between an insertion configuration wherein the second clipping member is axially separated from the first clipping member and a tissue-engaging configuration in which the second clipping member lockingly engages the cavity first clipping member to capture tissue therebetween.

14. The device of claim 13, wherein the first clipping member includes first and second slots extending thereinto from a rim thereof to define first and second deflectable regions.

15. The device of claim 13, wherein the second clipping member includes an elongated body extending from a first end to a second end and having a reduced diameter region formed between the first and second ends.

16. The device of claim 15, wherein the first end is threaded to engage a corresponding threading formed on an inner surface of the first clipping member.

17. The device of claim 15, wherein the first clipping member engages the second clipping member with a pressure-fit.

18. The device of claim 13, wherein, in the insertion configuration, the dome is invented so the rim is positioned proximally of the tip.

19. The device of claim 15, wherein the rim includes a radially protruding lip formed to lockingly engage the second end of the locking member.

20. A method for causing hemostasis, comprising:
advancing a first clipping member of a clipping device into a target portion of tissue, wherein the first clipping member is housed within an insertion device in a radially contracted insertion configuration and is radially expanded to a tissue engagement configuration upon release thereof from the insertion device;
advancing a second clipping member of the clipping device into the target portion of tissue, wherein second clipping member is housed within an insertion device in a radially contracted insertion configuration and is radially expanded to a tissue engagement configuration upon release thereof from the insertion device; and
moving the second clipping member toward the first clipping member along a longitudinal axis of the device to capture tissue therebetween.
